(19) United States
(12) Patent Application Publication
(10) Pub. No.: US 2009/0177092 A1
(43) Pub. Date: Jul. 9, 2009

(54) Noninvasive sensor system and method for detection of internal pathologic conditions

(76) Inventors: Ronald G. Riechers, Beavercreek, OH (US); Dennis Lee Allen, Beavercreek, OH (US); William P. Wiesmann, Washington, DC (US); Loland A. Pranger, Gaithersburg, MD (US)

Correspondence Address: LAW OFFICES OF BURTANEK & BHATTACHARYYA, LTD., 5575 STERRETT PLACE, SUITE 320, COLUMBIA, MD, 21044, US

(21) Appl. No.: 12/228,894

(22) Filed: Aug. 16, 2008

Related U.S. Application Data
(60) Provisional application No. 60/965,238, filed on Aug. 18, 2007.

(51) Int. Cl. A61B 8/00

(52) U.S. Cl. 600/459

(57) ABSTRACT

The present invention is directed to a system for non-invasively detecting the presence or absence of a trauma in a tissue region utilizing a dual-modality wand-detector. The wand integrates an electromagnetic transceiver and an ultrasound transducer to simultaneously obtain interrogation signatures from the tissue region by minimizing impedance mismatches that occur due to reflected energy at interfaces. Thereafter, the interrogation signals are processed in a signal processing system utilizing dual modality and impedance software to obtain trauma condition data that is subsequently displayed.

### PRIORITY INFORMATION

This application claims the benefit of priority from U.S. Provisional Application 60/965,238, filed Aug. 18, 2007.

### STATEMENT OF GOVERNMENT INTEREST

The United States Government as represented by the Secretary of Defense for the Defense Advanced Research Project Agency (DARPA) shall have a non-exclusive, non-transferable, irrevocable, paid up license to the subject invention.

### BACKGROUND OF THE INVENTION

Traumatic injury is a leading cause of death and disability, particularly amongst children and young adults in the U.S. Approximately 150,000 patients in this country die each year as a direct consequence of trauma. Even more astounding is that 400,000 patients are disabled every year. The cost for direct medical care of traumatically injured patients is estimated at more than $118 billion per year. In addition, it is estimated that 4 million potential years of productive life are lost annually due to these injuries. This exceeds heart disease, cancer and stroke combined.

Traumatic injuries are classified as either penetrating or blunt. Penetrating injuries disrupt the skin and underlying tissue and cause tissue disruption along the tract of the offending missile. All types of tissue, including skin, muscle, fascia, bone, blood vessels and organs, become involved. If severe, organ injury can lead to irreversible failure of function and death. Typically, penetrating injuries are caused by objects such as a knife or gunshot wounds.

Although blunt injuries do not violate the skin or tissue and are confined to superficial structures such as skin, muscle and small blood vessels, they may be as severe as penetrating injuries. Clinical manifestations range from bruising or small vascular bed disruption to the more severe bone and organ injury. An example of this type of trauma is a contusion from impact.

Blunt trauma injuries are frequently overlooked, as it is often difficult to determine the extent of injury based solely on visual inspection. Closer physical examination may be revealing but this is largely dependent on the expertise of the examiner, and a first responder may simply not have the clinical skills to make a diagnosis with a high degree of confidence. Additionally, conditions of examinations in pre-hospital settings often interfere with optimal physical examination.

The importance of early recognition cannot be overemphasized, especially in cases of intracranial damage. Efforts to improve diagnosis of these conditions have used sophisticated and noninvasive methods such as pulse oximetry, MRI, and ultrasound. However, these are not presently amenable to prehospital settings.

Detection of five serious traumatic conditions are often hampered by lack of diagnostic equipment or qualified diagnosticians. These include Pneumothorax, Hemothorax, Compartment Syndrome, Epidural Hematoma and Subdural Hematoma which are described below.

Pneumothorax

Pneumothorax is the collection of air or gas in the space around the lungs. The condition may result from chest trauma, excess pressure on the lungs, or lung disease such as COPD, asthma, cystic fibrosis, tuberculosis, or whooping cough. In some cases, the cause is unclear. Diagnostic indicators include decreased or no breath sounds on the affected side when listening through a stethoscope, chest x-ray to tell whether there is air outside the lung, and analysis of arterial blood gases. Up to 50% of patients who have a pneumothorax will have another, but there are no long-term complications after successful treatment.

Hemothorax

Hemothorax is a collection of blood in the space between the chest wall and the lung known as the pleural cavity. The most common cause of hemothorax is chest trauma. In blunt chest trauma, a rib may lacerate lung tissue or an artery, causing blood to collect in the pleural space. In penetrating chest trauma, a weapon such as a knife or bullet lacerates the lung. A large hemothorax will likely place the trauma victim in shock. Hemothorax may also be associated with pneumothorax. Depending on the amount of blood or air in the pleural cavity, a collapsed lung can lead to respiratory and hemodynamic failure known as tension pneumothorax. Diagnostic tests include decreased or absent breath sounds on the affected side, chest x-ray, thoracentesis, and pleural fluid analysis. The outcome depends on the underlying cause of the hemothorax and the promptness of the treatment. Possible complications are shock, fibrosis or scarring of the pleural membranes and death.

Compartment Syndrome

Thick layers of tissue called fascia separate groups of muscles in the arms and legs from each other. Inside each layer of fascia is a confined space, called a compartment, which includes the muscle tissue, nerves, and blood vessels (They are surrounded by the fascia much like wires surrounded by insulation.). Unlike a balloon, fascia do not expand, so any swelling in a compartment will lead to increasing pressure in that compartment, which will compress the muscles, blood vessels, and nerves. Compartment syndrome results from the compression of nerves and blood vessels within this enclosed space that leads to impaired blood flow and muscle and nerve damage. If this pressure is high enough, blood flow to the compartment will be blocked, which can lead to permanent injury to the muscle and nerves. If the pressure lasts long enough, the limb may die and need to be amputated. If the diagnosis of compartment syndrome is made promptly and surgical release performed, the outlook is excellent for recovery of the muscles and nerves inside the compartment. However, the overall prognosis will be determined by the injury leading to the syndrome. If there is a delay in diagnosis, there can be permanent nerve injury and loss of muscle function. This is more common when the injured person is unconscious or heavily sedated and incapable of complaining. Permanent nerve injury can occur after 12-24 hours of compression. Possible complications include permanent injury to nerves and muscles that can dramatically impair function. In more severe cases, limbs may need to be amputated because all the muscles in the compartment have died from lack of oxygen.

Epidural Hematoma

Epidural hematoma (also known as extradural hemorrhage or extradural hematoma) is bleeding between the inside of the skull and the outer covering of the brain called the dura. An epidural hematoma occurs when there is a rupture of a blood vessel, usually an artery, which then bleeds into the space between the dura mater and the skull. Extradural hemorrhages can also be caused by venous bleeding in young children. The affected vessels are often torn by skull fractures. This type of bleeding is more common in young people because the membrane covering the brain is not as firmly attached to the skull as it is in older people. Rapid bleeding causes a collection of blood known as hematoma. The hematoma presses on the brain causing a rapid increase in intracranial pressure which may then result in additional brain injury. Rapid diagnosis and treatment of epidural hematoma is critical as conditions can worsen in short periods of time that may result in permanent brain damage and death. A neurologic examination may indicate that a specific part of the brain is malfunctioning, as indicated by arm weakness on one side, or may indicate increased intracranial pressure. Increased cranial pressure will require emergency surgery to relieve the pressure within the head and spare the brain from further injury.

Subdural Hematoma

A subdural hematoma or subdural hemorrhage is a collection of blood between the dura and the brain when tiny veins between the surface of the brain and the dura stretch and tear, allowing blood to collect. A subdural hematoma can be caused by blunt trauma to the head. The elderly are particularly susceptible because the veins are often already stretched due to brain atrophy. Subdural hematomas can occur even after a very minor head injury and can go unnoticed resulting in chronic subdural hematomas. Some subdural hematomas occur without cause. Similar to epidural hematoma, emergency surgery may be needed to reduce pressure within the brain. Acute subdural hematomas occur when blood rapidly fills the brain area, limiting the space that the brain can occupy. The pressure from a subdural hematoma can further damage the central nervous system by decreasing blood flow to the brain or by causing the brain to herniate through the opening in the back of the skull where the spinal cord emerges from the skull. Acute subdural hematomas present the largest challenge with high rates of death and injury. Subacute and chronic subdural hematomas have good outcomes in most cases with symptoms going away after the blood collection is drained. There is a high frequency of seizures following a subdural hematoma, but these are usually well controlled with medication. Possible complications include temporary or permanent weakness, numbness, difficulty speaking, seizures, brain herniation, memory loss, dizziness, headache, anxiety, and difficulty concentrating. Thus, accurate diagnostics and treatment are critical.

The present invention overcomes drawbacks with prior art devices to rapidly detect trauma conditions as discussed hereinbelow.

### SUMMARY OF THE INVENTION

It is therefore an objective of the present invention to provide an integrated non-invasive electromagnetic and ultrasound trauma sensing and detection system for detecting traumatic injury in animals and humans.

It is also an objective of the present invention to provide an integrated non-invasive trauma sensing and detection system that includes a wand-detector subsystem, a processing and control subsystem and a display subsystem.

It is also an objective of the present invention to provide an integrated non-invasive trauma sensing and detection system that utilizes electromagnetic energy and ultrasound energy simultaneously.

It is also an objective of the present invention to provide an integrated non-invasive trauma sensing and detection system that is capable of impedance matching to minimize reflected signals and provide improved images of the trauma site.

It is also an objective of the present invention to provide a non-invasive electromagnetic sensing and detection system for detecting neurotrauma and brain seizure in animals and humans.

These and other objectives are discussed hereinbelow.

### DESCRIPTION OF THE DRAWINGS

FIG. 1 is a flow diagram of the system, DS.

FIG. 2 is a flow diagram of the wand-detector subsystem, W.

FIG. 2(a) is a top view of the wand-detector subsystem W.

FIG. 2(b) is an end view of the wand-detector subsystem W.

FIG. 2(c) is a schematic of the electromagnetic transceiver.

FIG. 2(d) is a schematic of the ultrasound transducer.

FIG. 3 is a flow diagram of the processor subsystem P

FIG. 4 is a flow diagram of the display subsystem D.

FIG. 5 is a flow diagram of the system DS with components.

FIG. 6(a) is a preferred embodiment of a modified radiator-receiver of the transceiver of wand-detector W.

FIG. 6(b) is another preferred embodiment of a modified radiator-receiver of the transceiver of wand-detector W.

FIG. 6(c) is another preferred embodiment of a modified radiator-receiver of the transceiver of wand-detector W.

FIG. 6(d) is another preferred embodiment of a modified radiator-receiver of the transceiver of wand-detector W.

FIG. 6(e) is another preferred embodiment of a modified transceiver of wand-detector W.

FIG. 6(f) is another preferred embodiment of a modified transceiver of wand-detector W.

FIG. 6(g) is another preferred embodiment of a modified transceiver of wand-detector W.

FIG. 6(h) is another preferred embodiment of a modified transceiver of wand-detector W.

FIG. 6(i) is another preferred embodiment of a modified transceiver of wand-detector W.

### DETAILED DESCRIPTION OF THE EMBODIMENTS

The present invention is directed to providing a system and method for non-invasively detecting internal pathologic injury resulting from conditions such as Pneumothorax, Hemothorax, Compartment Syndrome, Epidural Hematoma, Subdural Hematomas, Neuro-traumas, brain seizures, tumors of the brain and tumors of other tissues such as breast tissue. The system, in accordance with the present invention, simultaneously utilizes electromagnetic (EM) and ultrasound (US) modalities, the combination of which provides a composite constitutive parameter that is a mixture of the EM and the US modalities. This composite constitutive parameter is measurable from scattered energy when transmission signals propagate through a multi-layered material. The system of the present invention utilizes electrical permittivity for the EM aspect and density for the US aspect. The present invention may also utilize an integrated, non-invasive electromagnetic (EM) modality system for detecting neurotrauma, brain seizures and tumors.

The present invention is particularly directed to interrogating tissue regions to detect for the presence of traumas of the types discussed above. The overall tissue region includes a skin section, a fat section, a muscle section, a bone section and a target material section. For the purposes of this invention, the target material section is one or more sections of the tissue region that contains blood and/or air to indicate the presence of a trauma condition. Each of these sections exhibits a density and electrical permittivity that is different from the next adjacent section. The change in these constitutive properties creates an impedance mismatch at the interface between each section when interrogated by the system. Thus, when energy from the system is transmitted to the tissue region to be interrogated, mismatched impedance results in reflected energy at the interfaces and a concomitant loss of energy transmitted into the overall tissue region. The greater the difference in the electrical permittivity and density of the individual sections, the greater the range of values of the parameter in the overall tissue region. Additionally, as the proportion of one of the sections increases, so does its contribution to the overall constitutive property of the tissue region. Thus, analysis of transmitted and received signals by the system of the present invention provides information on the constitutive property of the overall tissue region and further provides information on the proportions of the individual sections. Additionally, the system transmits the maximum energy into the tissue region by matching the impedance of the first tissue section with the system's intrinsic impedance. In this manner, the system of the present invention minimizes diagnostic errors associated with section interface and provides accurate information on the presence or absence of trauma characteristics in the tissue region. These trauma characteristics may be displayed as images. The non-invasive trauma detecting and sensing system is discussed hereinbelow.

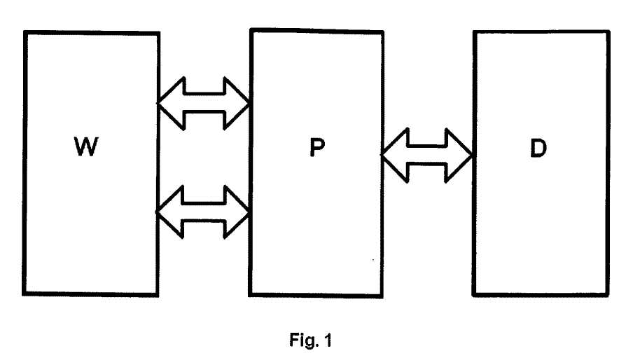

As shown in FIG. 1, the detecting and sensing system DS includes a wand-detector subsystem W, a processor and control subsystem P and a display subsystem D. Each of the subsystems are connected to one another by wired or wireless means. In a preferred embodiment, the system DS is integrated into a hand-held device.

### The Wand-Detector Subsystem, W

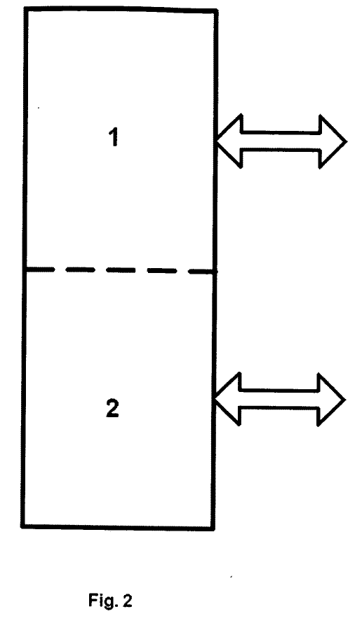

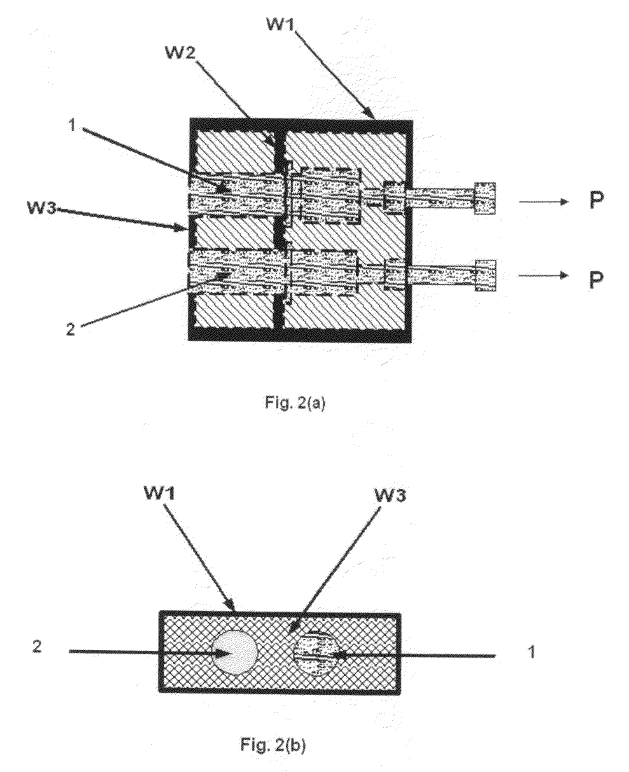

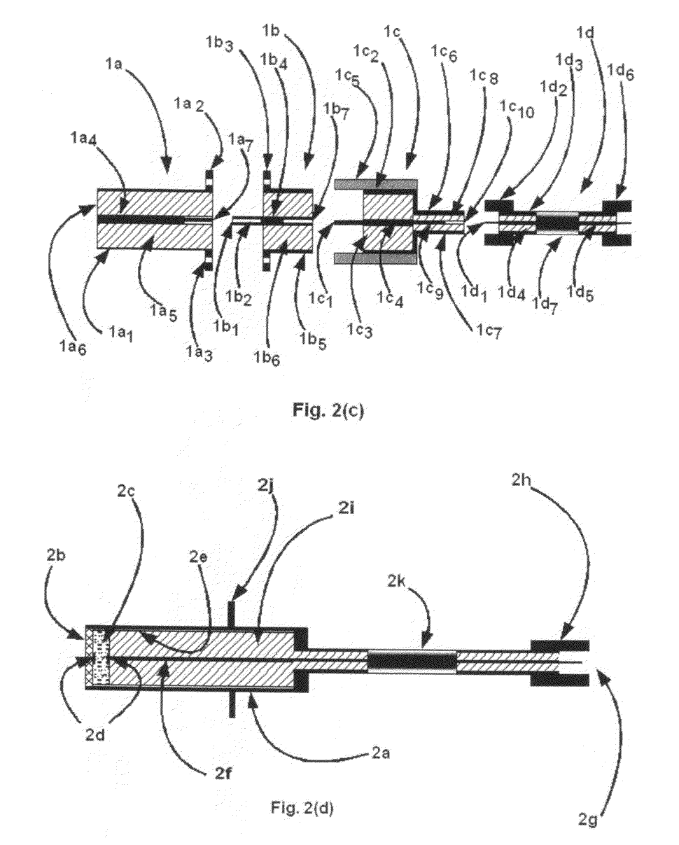

FIG. 2 is a flow diagram of the wand-detector system W including the transceiver 1 and the transducer 2. FIG. 2(a) shows a top view of the wand-detector subsystem W. FIG. 2(b) shows the end view of the wand-detector subsystem W. The wand-detector subsystem W includes an electromagnetic (EM) transceiver 1 and an ultrasonic (US) transducer 2, each having a preferred diameter of one-half inch. Both the transceiver 1 and the transducer 2 are hermetically sealed in a shock-resistant enclosure W₁. As shown in FIGS. 2(a) and 2(b), transceiver 1 and the transducer 2 are positioned in a fixed manner via intermediate plate W₂ and face plate W₃, to reduce registration error. Minimizing registration error significantly improves signal processing accuracy, which is critical to the operation of the system of the present invention. Additionally, the wand-detector W may include control/function switches and tactile feedback including but not limited to temperature and vibration sensors that allow for user input and/or feedback.

As discussed above, in order to transmit the maximum energy into the tissue region, the EM transceiver 1 of the present invention has an intrinsic impedance that matches the impedance of a first tissue section in contact with transceiver 1. Typically, the first tissue section is a skin section. The intrinsic impedance of the EM transceiver 1 is dependant upon the ratio of the inner diameter of the outer conductor (discussed below) and the diameter of the axial conductor (also discussed below), which is referred to as the aspect ratio. This aspect ratio is mathematically related to the intrinsic impedance of transceiver 1 as follows:

$${Z}_{0} \sqrt{\frac{μ}{ɛ}} ( \frac{ln ( \frac{b}{a} )}{2 π} ) \qquad ( 1 )$$

### Where:

Z₀ is the intrinsic impedance of the transceiver 1;
the permeability μ=μ₀μᵣ; μ₀=12.56637×10⁻⁷N/A²; μᵣ=1.0 for non-ferromagnetic materials;
the permittivity ε=ε₀εr; ε₀=8.85419×10⁻¹² F/m; εᵣ=2.1 for Teflon (the preferred material)

$${Z}_{0} = ( \frac{\sqrt{\frac{μ}{ɛ}}}{2 π} ) ln ( \frac{b}{a} ) = ( \frac{\sqrt{\frac{{μ}_{0} {μ}_{r}}{{ɛ}_{0} {ɛ}_{r}}}}{2 π} ) ln ( \frac{b}{a} ) = ( \frac{\sqrt{\frac{{μ}_{0}}{{ɛ}_{0}}} \sqrt{\frac{{μ}_{r}}{{ɛ}_{r}}}}{2 π} ) ln ( \frac{b}{a} ) = ( \frac{377 \sqrt{\frac{1}{2.1}}}{2 π} ) ln ( \frac{b}{a} ) \qquad ( 2 )$$

Computing the numerical coefficients yields:

$${Z}_{0} = 41.4 \times ln ( \frac{b}{a} ) \qquad ( 3 )$$

Additionally, the interrogating signal from transceiver 1 is affected by extraneous signals. Thereafter, above, the present invention provides an accurate estimation of a reflection coefficient, ρ, of an arbitrary load in terms of impedances of the transceiver 1 and those of the tissue region encountered during interrogation. The reflection coefficient, ρ is determined by the ratio of the difference between the impedance of the tissue region and the intrinsic impedance of the transceiver 1 to the sum of the impedance of the tissue region and the intrinsic impedance of transceiver 1.

The EM transceiver 1 as shown in FIG. 2(c) is capable of matching 50 ohm impedances. Transceiver 1 includes a radiator-receiver 1a, the bulkhead connector 1b, a cable adapter 1c, and a connector cable 1d. Radiator-receiver 1a includes a hollow cylindrical outer conductor 1a₁ which is fixedly connected at its proximal end to a flange 1a₂, having apertures 1a₃ through which fastening means (not shown) are utilized for fastening outer conductor 1a₁ to bulkhead connector 1b. The radiator-receiver 1a also includes a concentrically located straight axial conductor 1a₄ separated by a low-loss dielectric material 1a₅. The outer conductor 1a₁, the axial conductor 1a₄ and the dielectric material 1a₅ form an open-ended co-axial structure having a distal end surface 1a₆ that is in contact with the interrogated tissue region. The outer conductor 1a₁ the axial conductor 1a₄ and the dielectric material 1a₅ are maintained in a fixed position by attaching the outer conductor 1a₁ to flange 1a₂, preferably by soldering. Thereafter, the radiator receiver 1a is matingly connected to bulkhead connector 1b via pin 1a₇ that fits into the end 1b₁ of socket 1b₂ as shown. A flange 1b₃ is aligned with radiator-receiver 1a such that bulkhead connector 1b can be fastened to radiator-receiver 1a utilizing fastening means (not shown). An axial conductor 1b₄ is positioned between outer conductor 1b₅ and low-loss dielectric 1b₆. Outer conductor 1b₅ is fixed to flange 1b₃, preferably by soldering. The low-loss dielectric 1b₆ is fixedly positioned between the outer conductor 1b₃, and the socket 1b₂. The socket 1b₂ includes a second socket end 1b₇ that is utilized to matingly connect to pin 1c₁ of the cable adapter 1c. The cable adapter 1c also includes an outer conductor 1c₂, a low-loss dielectric material 1c₃ and an axial conductor 1c₄ that are maintained in a fixed manner so as to allow a housing 1c₅ to fit over outer conductor 1b₅ and rest at flange 1b₃ when bulkhead connector 1b is attached to cable adapter 1c. The cable adapter 1c also includes a reduced diameter portion 1c₆ having an outer conductor portion 1c₇, a low loss dielectric portion 1c₈ and an axial conductor portion 1c₉ each having a diameter that is proportionally smaller than that of the outer conductor 1c₂, low-loss dielectric 1c₃ and axial conductor 1c₄. The outer conductor portion 1c₇, low-loss dielectric portion 1c₈ and axial conductor portion 1c₉ are maintained in a fixed position. The reduced diameter portion 1c₆ allows for axial conductor 1c₄ to align with and contact conductor 1c₁ and for low loss dielectric 1c₃ to be aligned with and contact low-loss dielectric 1c₈. Axial conductor 1c₉ also includes a socket 1c₁₀ to matingly connect with pin 1d₁ of connector cable 1d. Connector cable 1d also includes a first connector portion 1d₂ that fits over the second outer conductor 1c₇ of cable adapter 1c when the adapter 1c is attached to connector cable 1d. Connector cable 1d also includes an outer conductor 1d₃, a low-loss dielectric 1d₄ and an axial conductor 1d₅ that are maintained in a fixed manner. The connector cable 1d includes a second connector portion 1d₆ that is connected to the control and processor subsystem P discussed below. The cable jacket 1d₇ surrounds the outer conductor 1d₃ to preserve the structural integrity of EM transceiver 1 and to protect transceiver 1 from environmental elements. It is important to note that the hollow outer conductors 1a₁ is aligned with hollow outer conductor 1b₅ which is further aligned with hollow outer conductor 1c₂ and 1c₇, which is further aligned with hollow outer conductor 1d₃ such that low-loss dielectrics 1a₅, 1b₆, 1c₃, 1c₈ and 1d₄ and the respective axial conductors 1a₄, 1b₄, 1c₄, 1c₉ and 1d₅ are aligned so as to allow the transceiver 1 to provide an integrated transmission line.

Low-loss dielectric 1a₅, 1b₆, 1c₃, 1c₈ and 1d₄ may be a solid, liquid or gas including, but not limited to, Teflon®, 90W oil or compatible gases although diameter adjustments would be required for impedance matching. Teflon® is preferred as the low-loss dielectric as it facilitates manufacture and retains constant permittivity with respect to frequency. Suitable material for the outer conductors 1a, 1b₅, 1c₂, 1c₇, 1d₃ and the axial conductors 1a₄, 1b₄, 1c₄, 1c₉ and 1d₅ include but are not limited to metals such as brass, copper, silver, gold and nanotubes. Other efficient electrical conductors are also within the scope of this invention.

As shown in FIG. 2(d), the US transducer 2 includes a housing 2a a wear plate 2b, an active piezoelectric element 2c, electrodes 2d, an inner sleeve 2e, a backing material 2f, a cable connector end 2g, a connector 2h, a second backing material 2i and a mounting flange 2j. The connector end 2g is utilized for connecting the transducer 2 to subsystem P via connector 2h. A cable jacket 2k preserves the structural integrity of transducer 2 and protects transducer 2 from environmental elements. Transducer 2 is utilized to measure the variations in echo delay from the time of transmission of the signal through the tissue region. These variations indicate acoustic impedance. The echo delays are captured as data samples that are utilized by processor P as discussed below. Additionally, the distance from the end of the transducer 2 to the interrogated tissue region is determined based on sampling frequency and the speed of sound in each section. A conventional off-the shelf transducer may be utilized.

In use, the transceiver 1 of the wand-detector W transmits electromagnetic waves that propagate through the tissue region, while transducer 2 of the wand-detector W simultaneously propagates acoustic waves through the same tissue region. With respect to the transceiver 1, reflected signals provide a measurement of the permittivity of each of these layers. In accordance with the present invention, the wand detector system W captures EM and US wave reflections as constitutive signatures of the individual tissue sections rather than a lumped boundary condition constitutive parameter. These signals are then transmitted to the processor P to determine the presence or absence of a trauma condition as discussed below.

The Processor and Control Subsystem P

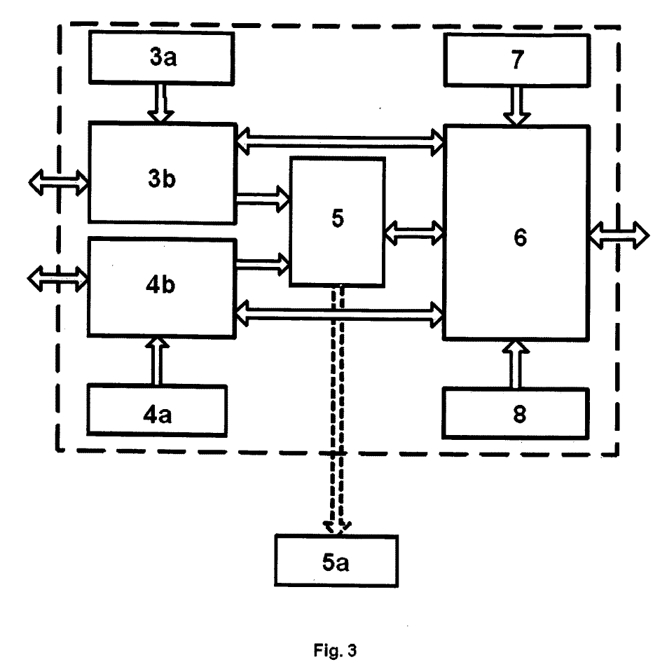

As shown in FIG. 3, the processor subsystem P includes an electromagnetic (EM) signal source 3a and an ultrasound (US) signal source 4a that generate electromagnetic and ultrasound interrogation signals. The signal from the EM signal source 3a is then conditioned in an EM front end and signal preprocessor 3b and then transmitted to the EM transceiver 1 of the wand W. Similarly, the signal from the US signal source 4a is then conditioned in an US front end and signal preprocessor 4b and then transmitted to US transducer 2 of the wand W. Upon interrogation of the tissue region utilizing the wand W, as discussed above, the EM signals from transceiver 1 and the US signals from transducer 2 are transmitted to the subsystem P digitized and conditioned prior to storage as raw data in data storage 5. In accordance with the present invention, the signals from the transceiver 1 and the transducer 2 are stored separately. The raw data may be stored in a non-volatile medium that is integral with the subsystem P. Additionally, raw data may also be stored in a remote data storage 5a utilizing wireless transmission mode or other well known means of transmission and storage. The EM and US data is simultaneously and separately transmitted to a central processing unit (CPU) 6. Also in accordance with the present invention the CPU 6 utilizes EM signature database 7 and US signature database 8 that provide data sets corresponding to various trauma conditions. Preferably, databases 7 and 8 store data on non-volatile medium that is integral to the subsystem P. Trauma signatures from databases 7 and 8 are processed in CPU 6 along with the signals obtained from transceiver 1 and transducer 2 as discussed above. In accordance with the present invention, the CPU 6 utilizes the transceiver 1 and transducer 2 data and the data from databases 7 and 8 and processes the signals utilizing a combination of software that utilizes one-dimensional resolution analysis for the dual modalities to determine the presence or absence of trauma characteristics. The data samples obtained from transducer 2 are converted to real time scale for known sampling frequencies. The sampling frequencies and available information on the speed of sound within each tissue section may be utilized in determining optimal positioning of the transducer 2 of wand-detector W at the tissue region. Additionally, the sampling frequencies and speed of sound information provides information on echo reflection peaks and acoustic impedance mismatches at the section interfaces. Also in accordance with the present invention, the resultant data may be obtained in an intermediate form so as to allow for further medical analysis. This intermediate data may be stored within the system DS or transmitted to a remote storage means (not shown). Alternatively, based pm the degree of correspondence between the trauma signature and the reflections detected from the tissue region, the present invention also provides that the system DS is capable of making a recommendation within specified confidence limits to the operator for triage decision support. Recommendations may include alternatives such as trauma detected, indeterminate, poor sensor placement or no significant trauma detected.

The Display Subsystem D

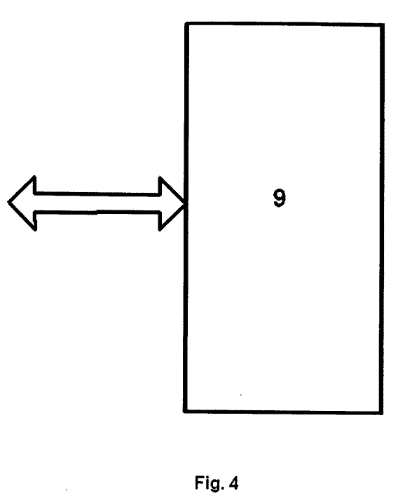

As shown in FIG. 4, the Display Subsystem D includes a display monitor 9 having at least one of audio and visual capabilities to display relevant information obtained from the wand-detector W and the processor and control P. In accordance with the present invention monitor 9 may include a user interface for controlling display characteristics such as screen contrast and brightness as well as operational directions for the wand-detector W via the processor and control P. Alternatively, or as supplementary manual control, the display 9 may include button switches (not shown) to actuate predefined functions as well as user input capabilities. Pertinent information displayed on monitor 9 includes, but is not limited to system DS power level, current time and date, patient identification, available memory, and auditory and visual alerts for indeterminate patients states an alert after a specified elapsed time since the last reading as well as the total time since Headphones (not shown) may be utilized with the subsystem D to enable silent operation.

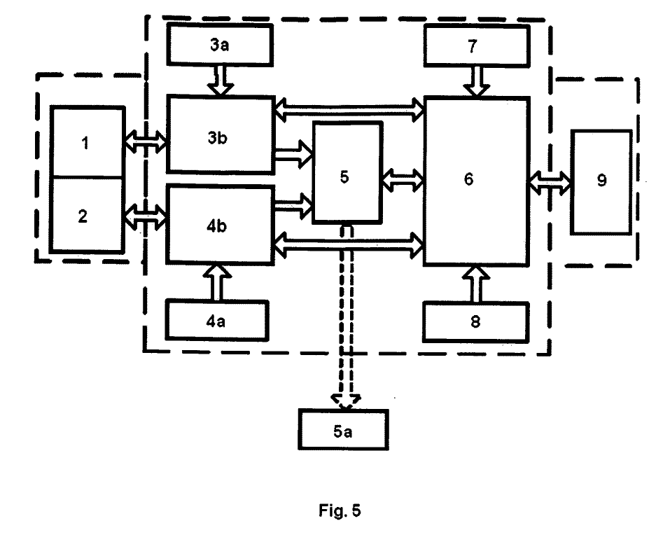

The overall system DS with the components of subsystems W, P and D are as shown in FIG. 5. These subsystems and components are discussed above. It is important to note that the system DS may be integrated into a one-piece hand held device or may be a multi-component system that is capable of withstanding environmental stresses. The following preferred embodiments provide alternate radiator-receivers or transceivers to optimize subsystem W as discussed below.

### Preferred Embodiments

The following preferred embodiments of the radiator-receiver and the transceiver provide optimal impedance matching for impedances equal to 50 ohms, greater than 50 ohms and less than 50 ohms as discussed below and determined by equations (1), (2), (3), discussed above. Additionally, the preferred embodiments maximize transmission of signals with minimal energy loss.

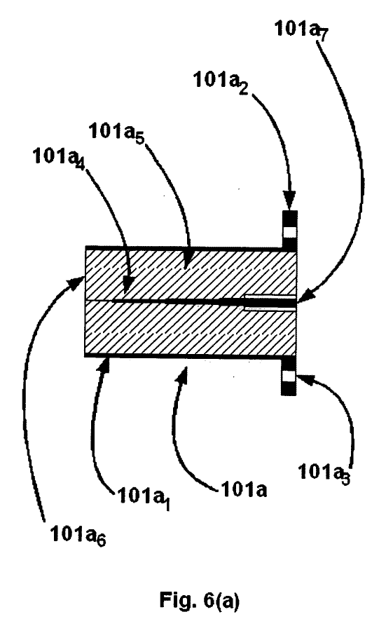

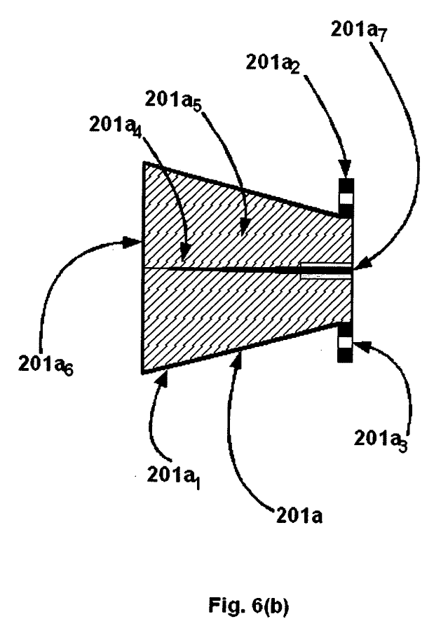

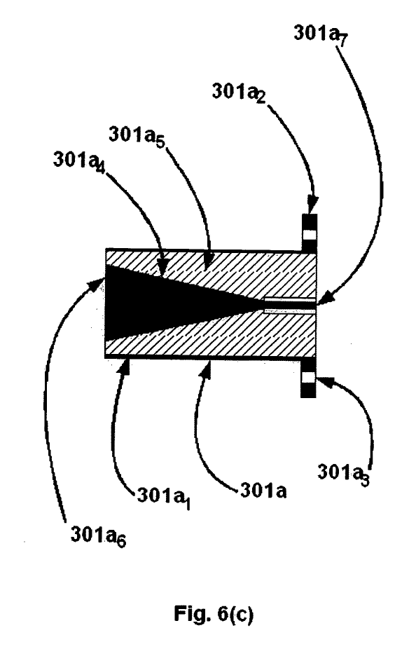

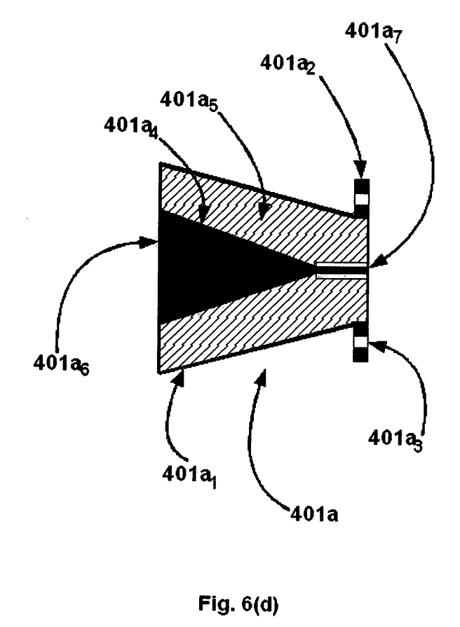

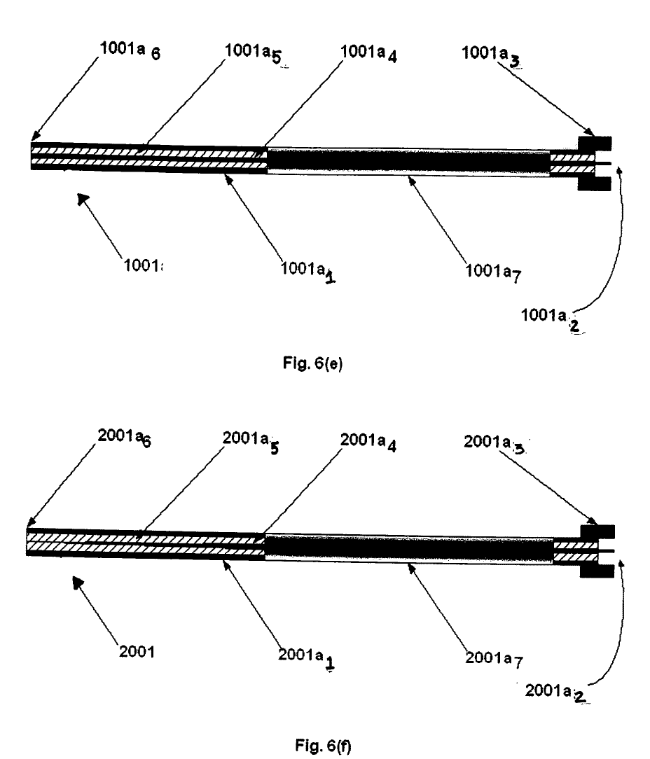

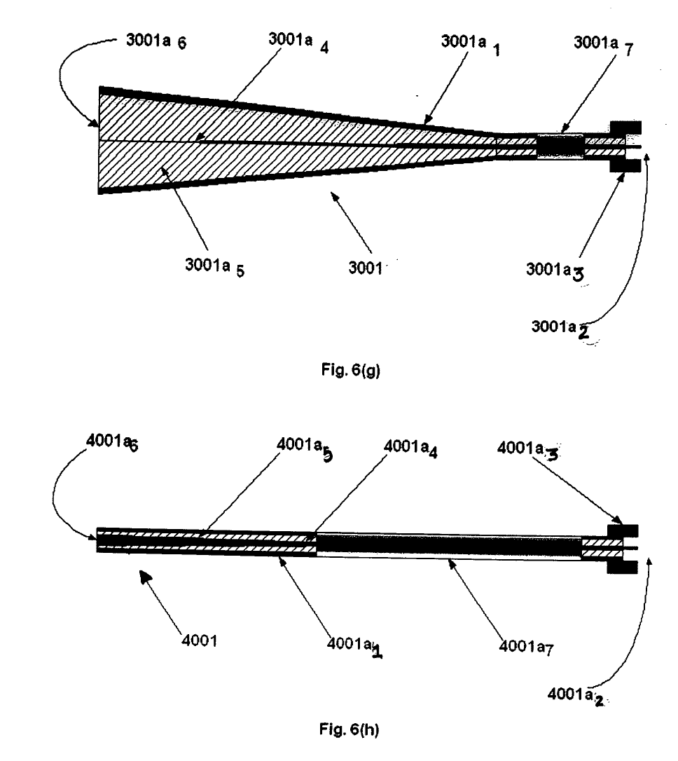

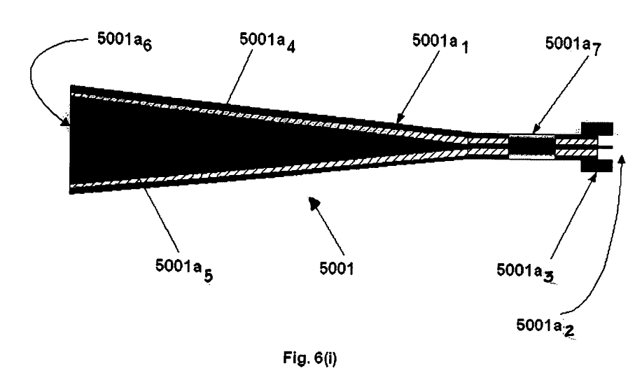

A preferred embodiment of transceiver 1 includes a modified radiator-receiver 101a as shown in FIG. 6(a). The modified radiator-receiver 101a may be utilized in place of radiator-receiver 1a, discussed above. The preferred transceiver is an open-ended coaxial structure where radiator-receiver 101a is capable of matching impedances greater than 50 ohms. The radiator-receiver 101a includes a hollow outer conductor 101a₁ that is fixedly connected at its proximal end to a flange 101a₂, having apertures 101a₃ for fastening to bulkhead connector 1b using fastening means. The radiator-receiver 101a also includes a tapered axial conductor 101a₄ separated by a low-loss dielectric material 101a₅. The outer conductor 101a₁, the axial conductor 101a₄ and the dielectric material 101a₅ form an open-ended coaxial structure having a distal end surface 101a₆ that is in contact with the tissue region being interrogated. The outer conductor 101a₁ the tapered axial conductor 101a₄ and the dielectric material 101a₅ are maintained in a fixed position by attaching the outer conductor 101a₁ to flange 101a₂, preferably by soldering. The tapered axial conductor 101a₄ has a continuously increasing diameter from the distal end 101a₆ to the proximal end in connection with bulkhead connector 1b (not shown). In accordance with the present invention, the diminishing ratio between the constant inner diameter of hollow outer conductor 101a₁ and the continuously increasing diameter of axial conductor 101a₄ acts to continuously decrease component impedance of the transmission line thereby allowing enhanced impedance matching between the detector system and the tissue region being interrogated.

Another preferred embodiment of transceiver 1 includes a modified radiator-receiver 201a as shown in FIG. 6(b). The modified radiator-receiver 201a may be utilized in place of radiator-receiver 1a, discussed above. The transceiver 1 is an open-ended coaxial structure where the radiator-receiver 201a is capable of matching impedances greater than 50 ohms. The radiator-receiver 201a includes a hollow, flared outer conductor 201a₁ that is fixedly connected at its proximal end to a flange 201a₂, having apertures 201a₃ for fastening to bulkhead connector 1b (not shown) using fastening means. The radiator-receiver 201a also includes a tapered axial conductor 201a₄ separated by a low-loss dielectric material 201a₅. The outer conductor 201a₁, the axial conductor 201a₄ and the dielectric material 201a₅ form an open-ended coaxial structure having distal end surface 201a₆ that is in contact with the tissue region being interrogated. The outer conductor 201a₁ the tapered axial conductor 201a₄ and the dielectric material 201a₅ are maintained in a fixed position by attaching the outer conductor 201a₁ to flange 201a₂, preferably by soldering. The tapered axial conductor 201a₄ has a continuously increasing diameter from the distal end 201a₆ to the proximal end in connection with bulkhead connector 1b (not shown). In accordance with the present invention, the aspect ratio of the inner diameter of the continuously flared hollow conductor 201a₁ to the continuously increasing diameter of axial conductor 201a₄ acts to continuously decrease component impedance of the transmission line thereby allowing enhanced impedance matching between the detector system and the tissue region being interrogated.

Another preferred embodiment of transceiver 1 includes a modified radiator-receiver 301a as shown in FIG. 6(c). The modified radiator-receiver 301a may be utilized in place of radiator-receiver 1a, discussed above. The transceiver 1 is an open-ended coaxial structure where the radiator-receiver 301a is capable of matching impedances less than 50 ohms. The radiator-receiver 301a includes a hollow outer conductor 301a₁ that is fixedly connected at its proximal end to a flange 301a₂, having apertures 301a₃ for fastening to bulkhead connector 1b (not shown) using fastening means. The radiator-receiver 301a also includes a flared axial conductor 301a₄ separated by a low-loss dielectric material 301a₅. The outer conductor 301a₁, the axial conductor 301a₄ and the dielectric material 301a₅ form an open-ended coaxial structure having distal end surface 301a₆ that is in contact with the tissue region being interrogated. The outer conductor 301a₁, the flared axial conductor 301a₄ and the dielectric material 301a₅ are maintained in a fixed position by attaching the outer conductor 301a₁ to flange 301a₂, preferably by soldering. The flared axial conductor 301a₄ has a continuously decreasing diameter from the distal end 301a₆ to the proximal end in connection with bulkhead connector 1b. In accordance with the present invention, the aspect ratio for the inner diameter of hollow outer conductor 301a₁ and the continuously decreasing diameter of axial conductor 301a₄ acts to continuously decrease component impedance of the transmission line thereby allowing for enhanced impedance matching between the detector system and the tissue region being interrogated.

Another preferred embodiment of transceiver 1 includes a modified radiator-receiver 401a as shown in FIG. 6(d). The modified radiator-receiver 401a may be utilized in place of radiator-receiver 1a, discussed above. The transceiver 1 is an open-ended coaxial structure where the radiator-receiver 401a is capable of matching impedances less than 50 ohms. The radiator-receiver 401a includes a hollow flared outer conductor 401a₁ that is fixedly connected at its proximal end to a flange 401a₂, having apertures 401a₃ for fastening to bulkhead connector 1b (not shown) using fastening means. The radiator-receiver 401a also includes a flared axial conductor 401a₄ separated by a low-loss dielectric material 401a₅. The outer conductor 401a₁, the axial conductor 401a₄ and the dielectric material 401a₅ form an open-ended coaxial structure having distal end surface 401a₆ that is in contact with the tissue region being interrogated. The flared outer conductor 401a₁ the flared axial conductor 401a₄ and the dielectric material 401a₅ are maintained in a fixed position by attaching the outer conductor 401a₁ to flange 401a₂, preferably by soldering. The flared outer conductor 401a₁ and the flared axial conductor 401a₄ have continuously decreasing diameters from the distal end 401a₆ to the proximal end in connection with bulkhead connector 1b (not shown). In accordance with the present invention, the aspect ratio between the constant inner diameter of flared hollow outer conductor 401a₁ and the flared diameter of axial conductor 401a₄ acts to continuously increase component impedance of the transmission line thereby allowing enhanced impedance matching between the detector system and the tissue region being interrogated.

Another preferred embodiment of the present invention, as shown in FIG. 6(e), provides an integrated open-ended coaxial EM transceiver 1001 capable of matching impedances equal to 50 ohms. Transceiver 1001 may be utilized in place of transceiver 1, discussed above. Transceiver 1001 includes a hollow outer conductor 1001a₁, a proximal end 1001a₂ having a connector 1001a₃ matingly connected to the control and processing subsystem P (not shown), an axial conductor 1001a₄, a low-loss dielectric material 1001a₅, a distal end 1001a₆ that is in contact with the tissue region being interrogated and a cable jacket 1001a₇. The outer conductor 1001a₁ the axial conductor 1001a₄ and the dielectric material 1001a₅ are maintained in a fixed position. The integrated EM transceiver 1001 minimizes energy loss that occurs at connections and maintains an aspect ratio that minimizes impedance mismatch. The cable jacket 1001a₇ surrounds the outer conductor 1001a₁ to preserve the structural integrity of EM transceiver 1001 and to protect transceiver 1001 from environmental elements.

Another preferred embodiment of the present invention, as shown in FIG. 6(f), provides an integrated open-ended coaxial EM transceiver 2001 capable of matching impedances greater than 50 ohms. Transceiver 2001 may be utilized in place of transceiver 1, discussed above. Transceiver 2001 includes a hollow outer conductor 2001a₁, a proximal end 2001a₂ having a connector 2001a₃ matingly connected to the control and processing subsystem P (shown above), a tapered axial conductor 2001a₄, a low-loss dielectric material 2001a₅, a distal end 2001a₆ that is in contact with the tissue region being interrogated and a cable jacket 2001a₇. The outer conductor 2001a₁ the tapered axial conductor 2001a₄ and the dielectric material 2001a₅ are maintained in a fixed position. The tapered axial conductor 2001a₄ has a continuously increasing diameter from the distal end 2001a₆ to the proximal end 2001a₂ in connection with processor P (not shown). In accordance with the present invention, the diminishing ratio between the constant inner diameter of hollow outer conductor 2001a₁ and the continuously increasing diameter of axial conductor 2001a₄ acts to continuously decrease component impedance of the transmission line. The integrated EM transceiver 2001 minimizes energy loss that occurs at connections and maintains an aspect ratio that minimizes impedance mismatch. The cable jacket 2001a₇ surrounds the outer conductor 2001a₁ to preserve the structural integrity of EM transceiver 2001 and to protect transceiver 2001 from environmental elements.

Another preferred embodiment of the present invention, as shown in FIG. 6(g), provides an integrated open-ended coaxial EM transceiver 3001 that is capable of matching impedances greater than 50 ohms. Transceiver 3001 may be utilized in place of transceiver 1, discussed above. Transceiver 3001 includes a hollow flared outer conductor 3001a₁, a proximal end 3001a₂ having a connector 3001a₃ matingly connected to the control and processing subsystem P (not shown), a tapered axial conductor 3001a₄, a low-loss dielectric material 3001a₅, a distal end 3001a₆ that is in contact with the tissue region being interrogated and a cable jacket 3001a₇. The tapered axial conductor 3001a₄ has a continuously increasing diameter from the distal end 3001a₆ to the proximal end 3001a₂ that is in connection with subsystem P (not shown). The flared outer conductor 3001a₁ the tapered axial conductor 3001a₄ and the dielectric material 3001a₅ are maintained in a fixed position. The integrated EM transceiver 3001 minimizes energy loss that occurs at connections and maintains an aspect ratio that minimizes impedance mismatch. The cable jacket 3001a₇ surrounds the outer conductor 3001a₁ to preserve the structural integrity of EM transceiver 3001 and to protect transceiver 3001 from environmental elements.

Another preferred embodiment of the present invention, as shown in FIG. 6(h), provides an integrated open-ended coaxial EM transceiver 4001 that is capable of matching impedances less than 50 ohms. Transceiver 4001 may be utilized in place of transceiver 1, discussed above. Transceiver 4001 includes a hollow outer conductor 4001a₁, a proximal end 4001a₂ having a connector 4001a₃ matingly connected to the control and processing subsystem P (not shown) a flared axial conductor 4001a₄, a low-loss dielectric material 4001a₅, a distal end 4001a₆ that is in contact with the tissue region being interrogated and a cable jacket 4001a₇. The flared axial conductor 4001a₄ has a continuously decreasing diameter from the distal end 4001a₆ to the proximal end 4001a₂ in connection with processor subsystem P (not shown). The outer conductor 4001a₁ the flared axial conductor 4001a₄ and the dielectric material 4001a₅ are maintained in a fixed position. The integrated EM transceiver 4001 minimizes energy loss that occurs at connections and maintains an aspect ratio that minimizes impedance mismatch. The cable jacket 4001a₇ surrounds the outer conductor 4001a₁ to preserve the structural integrity of EM transceiver 4001 and to protect transceiver 4001 from environmental elements.

Another preferred embodiment of the present invention, as shown in FIG. 6(i), provides an integrated open-ended coaxial EM transceiver 5001 that is capable of matching impedances less than 50 ohms. Transceiver 5001 may be utilized in place of transceiver 1, discussed above. Transceiver 5001 includes a hollow flared outer conductor 5001a₁, a proximal end 5001a₂ having a connector 5001a₃ matingly connected to the control and processing subsystem P (not shown), a flared axial conductor 5001a₄, a low-loss dielectric material 5001a₅, a distal end 5001a₆ that is in contact with the tissue region being interrogated and a cable jacket 5001a₇. The outer conductor 5001a₁ the flared axial conductor 5001a₄ and the dielectric material 5001a₅ are maintained in a fixed position. The flared axial conductor 5001a₄ has a continuously decreasing diameter from the distal end 5001a₆ to the proximal end 5001a₂ that is in connection with subsystem P (not shown). The integrated EM transceiver 5001 minimizes energy loss that occurs at connections and maintains an aspect ratio that minimizes impedance mismatch. The cable jacket 5001a₇ surrounds the outer conductor 5001a₁ to preserve the structural integrity of EM transceiver 5001 and to protect transceiver 5001 from environmental elements.

Low-loss dielectric 101a₅, 201a₅, 301a₅, 401a₅, 1001a₅, 2001a₅, 3001a₅, 4001a₅ and 5001a₅ may be a solid, liquid or gas including, but not limited to, Teflon®, 90W oil or compatible gases although diameter adjustments would be required for impedance matching. Teflon® is preferred as the low-loss dielectric as it facilitates manufacture and retains constant permittivity with respect to frequency. Suitable material for the outer conductors 101a₁, 201a₁, 301a₁, 401a₁, 1001a₁, 2001a₁, 3001a₁, 4001a₁, 5001a₁ and the axial conductors 101a₄, 201a₄, 301a₄, 401a₄, 1001a₄, 2001a₄, 3001a₄, 4001a₄, 5001a₄ include but are not limited to metals such as brass, copper, silver, gold, and nanotubes. Other efficient electrical conductors are also within the scope of this invention. Based on the preferred embodiment chosen, it is important to note that the hollow outer conductors 101a₁, 201a₁, 301a₁, 401a₁ are each aligned with hollow outer conductor 1b₅ which is further aligned with hollow outer conductor 1c₂ and 1c₇, which is further aligned with hollow outer conductor 1d₃ such that low-loss dielectrics 101a₅, 201a₅, 301a₅, 401a₅ corresponding to the preferred outer conductor listed above, and axial conductors 101a₄, 201a₄, 301a₄, 401a₄ corresponding to the preferred outer conductor and low-loss dielectric, are aligned so as to allow the transceiver 1, 1001, 2001, 3001, 4001 and 5001 to provide an integrated transmission line.

In operation, the system DS utilizes EM transceiver 1, 1001, 2001, 3001, 4001 or 5001 to transmit an EM signal, depending upon the tissue region impedance. As will be understood by one of ordinary skill, transceiver 1 includes radiator-receiver embodiments 101a, 201a, 301a, and 401a. As will also be understood by one of ordinary skill, known impedances of tissue regions are utilized to determine which of the transceivers 1, 1001, 2001, 3001, 4001 or 5001 shall provide optimal results. Thus, transceiver 1, 1001, 2001, 3001, 4001 or 5001 transmits EM signals obtained from the signal source 3a. The transducer 2 simultaneously transmits an US signal obtained from the signal source 4a to the tissue region being interrogated. The signals are partially reflected at the interfaces at the individual layers, and partially propagated through the tissue region. These signals are received by transceiver 1 or the other preferred transceiver embodiments, and transducer 2, then processed in subsystem P. The processed signals are then displayed in subsystem D, also, as discussed above.

What is claimed is:

1. A non-invasive trauma sensing and detection system comprising:
a wand-detector subsystem, a processor and control subsystem and a display subsystem;
said wand-detector further comprising an electromagnetic transceiver and an ultrasound transducer;
said transceiver constructed so as to transmit and receive electromagnetic signals from a tissue region having a plurality of tissue sections, wherein said sections each have interfaces;
said transducer constructed so as to transmit and receive acoustic signals from said tissue region;
said transceiver and said transducer constructed so as to operate simultaneously, said system constructed to as to minimize diagnostic errors associated with impedance mismatch between said wand-detector subsystem and said tissue region;
said system further constructed so as to detect for the presence of trauma signatures within said tissue region.

2. A detection system as recited in claim 1 wherein said wand-detector subsystem is constructed so as to position said electromagnetic transceiver and said ultrasonic transducer in a fixed manner within a housing;
said housing further comprising an intermediate plate and a face plate, said intermediate plate and said face plate constructed so as to maintain spacing between said transceiver and said transducer and minimize registration error;
said wand-detector subsystem further comprising an enclosure for hermetically sealing said transceiver and said transducer within said housing of said wand-detector subsystem so as to minimize environmental shocks;
said wand-detector further constructed so as to simultaneously propagate said electromagnetic and said ultrasound signals so as to form constitutive signatures of each of the tissue sections and transmit said signals to said processor subsystem.

3. A detection system as recited in claim 2, wherein said ultrasound transducer comprises a housing including a wear plate connected to an active piezoelectric element, electrodes, an inner sleeve, a first backing material, a cable connector end, a connector, a second backing material, and a mounting flange;
said cable connector end constructed so as to connect said transducer to said processor subsystem;
a cable jacket constructed so as to maintain said transducer structural integrity and protect said transducer from environmental elements;
said transducer constructed so as to transmit said ultrasound interrogation signals, measure variations in echo delay from a transmission time through said tissue region and further constructed so as to capture said echo delays as data sample;
said transducer further constructed so as to transmit said data samples to said processor subsystem.

4. A detection system as recited in claim 3, wherein said processor subsystem comprises an electromagnetic signal source constructed so as to generate electromagnetic interrogation signals, said electromagnetic signal source constructed so as to connect to an electromagnetic front end and signal preprocessor, said electromagnetic front end and preprocessor constructed so to condition said electromagnetic interrogation signal, said electromagnetic preprocessor further constructed so as to connect to said electromagnetic transceiver such that said generated electromagnetic interrogation signals are transmitted to said electromagnetic transceiver;
an ultrasound signal source constructed so as to generate ultrasound interrogation signals, said ultrasound signal source further constructed so as to connect to an ultrasound frond end and signal preprocessor, said preprocessor constructed so as to condition said ultrasound interrogation signals and further constructed so as to connect to said ultrasound transducer such that said generated ultrasound interrogation signals are transmitted to said ultrasound transducer;
said processor subsystem further constructed so as to receive electromagnetic signals from said transceiver and ultrasound signals from said transducer, subsequent to interrogation of said tissue region;
a data storage constructed so as to store said received electromagnetic signals and said received ultrasound signals as raw electromagnetic interrogation signals;
said processor subsystem further constructed so as to store said ultrasound interrogation signals as raw ultrasound interrogation signals in said data storage;
said processor subsystem further constructed so as to store said raw electromagnetic signals and said ultrasound interrogation signals separately;
said processor subsystem further constructed so as to simultaneously and separately transmit said raw electromagnetic interrogation signals and said raw ultrasound interrogation signals from said data storage to a central processing unit;
an electromagnetic signature database constructed so as to provide a first data set corresponding to trauma conditions to said computer processing unit;
an ultrasound signature database constructed so as to provide a second data set corresponding to said trauma conditions to said computer processing unit;
said computer processing unit further constructed so as to process said electromagnetic interrogation signals and said ultrasound interrogation signals utilizing dual modality and impedance matching software and provide electromagnetic signatures;
said processor subsystem further constructed so as to analyze said data to determine said presence of trauma within said tissue region.

5. A detection system as recited in claim 4 wherein said system further comprises
a display monitor constructed so as to provide at least one of audio and visual capabilities of information obtained from said wand-detector and said processor subsystem;
said display monitor further comprising an user-interface constructed so as to control display characteristics and input operational directions;
said display monitor further constructed so as to display data from said wand-detector and said processor subsystem.

6. A detection system as recited in claim 5 wherein said data storage comprises a remote data storage, said processor subsystem constructed so as to transmit said data to said remote data storage;
said data storage, said electromagnetic data from said electromagnetic signature database and said ultrasound data from said ultrasound signature database further comprising a non-volatile medium.

7. A detection system as recited in claim 6 wherein said transceiver comprises a radiator-receiver connected to a bulkhead connector, a cable adapter and a connector cable, said radiator-receiver having a first hollow outer conductor, a first axial conductor positioned within said hollow outer conductor and separated from said first axial conductor by a first low-loss dielectric.

8. A detection system as recited in claim 7, wherein said bulkhead connector further comprises:
a socket having a first end so as to matingly connect said radiator-receiver in said bulkhead connector;
a flange constructed so as to align with said radiator receiver and fasten said bulkhead connector to said radiator-receiver;
a second axial conductor positioned between a second outer conductor and a second low-loss dielectric, said outer connector fixed to said flange;
a second socket end of said socket further constructed so as to connect to said cable adapter.

9. A detection system as recited in claim 8, wherein said cable adapter further comprises:
a pin constructed so as to connect said cable adapter to said second socket end;
a third outer conductor, a third low-loss dielectric material and a third axial conductor constructed so as to be maintained in a fixed manner and allow a housing to fit over said second outer conductor and rest at said flange when said bulkhead connector is attached to said cable adapter;
a reduced diameter portion having a third outer conductor portion, a third low-loss dielectric portion and a third axial conductor portion, said third outer conductor portion, said third low-loss dielectric portion and third axial conduction portion constructed so as to each have a diameter that is proportionally smaller than that of said third outer conductor, said third low-loss dielectric and third axial conductor;
said third outer conductor portion, said third low-loss dielectric portion and said third axial conductor portion maintained in a fixed position and further constructed so as to allow said third axial conductor to align with and contact said third axial conductor portion, said third low-loss dielectric to be aligned with and contact said third low-loss dielectric portion;
said third axial conductor portion further constructed so as to matingly connect with said connector cable.

10. A detection system as recited in claim 9, wherein said connector cable further comprises:
a pin so as to matingly connect said connector cable with an aperture in said axial conductor;
a first connector portion constructed so as to fits over said third outer conductor portion of said cable adapter when said adapter is attached to said connector cable;
a fourth outer conductor, a fourth low-loss dielectric and a fourth axial conductor constructed so as to be maintained in a fixed manner;
a second connector portion constructed so as to connect with said control and processor subsystem;
a cable jacket constructed so as to surround said fourth outer conductor and further constructed so as to preserve said transceiver structural integrity and to protect said transceiver from environmental elements.

11. A detection system as recited in claim 10 wherein said low-loss dielectric comprises Teflon.

12. A detection system as recited in claim 11, wherein said outer conductors and said axial conductors further comprise a material selected from the group consisting of brass, copper, silver gold or nanotubes.

13. A detection system as recited in claim 12, wherein said radiator-receiver comprises:
said first hollow cylindrical outer conductor having a proximal end, said first hollow conductor constructed so as to fixedly connect at its proximal end to a flange so as to connect said conductor to said bulkhead connector;
said first straight axial conductor constructed to as to be positioned concentrically within said first outer conductor and separated by said first low-loss dielectric, said outer conductor said first axial conductor and said first low-loss dielectric material constructed so as to form an open-ended co-axial structure having a distal end surface, said distal end surface of said co-axial structure constructed so as to be in contact with said tissue region being interrogated; and
said radiator-receiver constructed so as to match 50 ohm impedances; and
wherein said first outer conductor having an inner diameter and said first axial conductor having a diameter, said inner diameter and diameter forming an aspect ratio, said aspect ratio providing a continuously decreasing component impedance of a transmission line and allowing enhanced impedance matching between said detector system and said tissue region being interrogated.

14. A detection system as recited in claim 12, wherein said radiator-receiver comprises a first axial conductor that is tapered so as to have a continuously increasing diameter from a distal end to a proximal end and positioned within said first outer conductor such that said first axial conductor said first low-loss dielectric material and said first outer conductor form an open-ended coaxial structure constructed so as to be in contact with the said tissue region being interrogated at its distal end;
said first axial conductor, said first outer conductor and said low-loss dielectric material are maintained in a fixed position;
said radiator-receiver constructed so as to connect to said bulkhead connector; and
said radiator-receiver constructed so as to match impedances greater than 50 ohms;
wherein said first outer conductor having an inner diameter and said first axial conductor having a diameter, said inner diameter and said diameter forming an aspect ratio, said aspect ratio providing a continuously decreasing component impedance of a transmission line and allowing enhanced impedance matching between said detector system and said tissue region being interrogated

15. A detection system as recited in claim 12 wherein said radiator-receiver comprises a hollow, flared first outer conductor, a tapered first axial conductor, said first outer conductor said tapered axial conductor and said low-loss dielectric constructed so as to form an open ended co-axial structure having a distal end that is in contact with said tissue region being interrogated; said tapered first axial conductor having a continuously increasing diameter from said distal end to a proximal end constructed so as to connect to said bulkhead connector;
said radiator-receiver constructed so as to match impedances greater than 50 ohms; and
wherein said first outer conductor having an inner diameter and said first axial conductor having a diameter, said inner diameter and said diameter forming an aspect ratio, said aspect ratio providing a continuously increasing component impedance of a transmission line and allowing enhanced impedance matching between said detector system and said tissue region being interrogated.

16. A detection system as recited in claim 12 wherein said radiator-receiver comprises a flared first axial conductor, said first outer conductor said flared axial conductor and said low-loss dielectric constructed so as to form an open ended co-axial structure having a distal end that is in contact with said tissue region being interrogated;
said flared first axial conductor having a continuously decreasing diameter from said distal end to a proximal end constructed so as to connect to said bulkhead connector;
said radiator-receiver constructed so as to match impedances less than 50 ohms; and
wherein said first outer conductor having an inner diameter and said first axial conductor having a diameter, said inner diameter and said diameter forming an aspect ratio, said aspect ratio providing a continuously decreasing component impedance of a transmission line and allowing enhanced impedance matching between said detector system and said tissue region being interrogated.

17. A detection system as recited in claim 12, wherein said radiator-receiver comprises
A hollow flared first outer conductor, a flared first axial conductor, said first outer conductor said tapered axial conductor and said low-loss dielectric constructed so as to form an open ended co-axial structure having a distal end that is in contact with said tissue region being interrogated;
said flared outer conductor and said flared first axial conductor having a continuously decreasing diameter from said distal end to a proximal end constructed so as to connect to said bulkhead connector;
said radiator-receiver constructed so as to match impedances less than 50 ohms; and
wherein said first outer conductor having an inner diameter and said first axial conductor having a diameter, said inner diameter and said diameter forming an aspect ratio, said aspect ratio providing a continuously increasing component impedance of a transmission line and allowing enhanced impedance matching between said detector system and said tissue region being interrogated.

18. A detection system as recited in claim 6 wherein said transceiver comprises:
a hollow outer conductor having a distal end and a proximal end, said proximal end connected to said control and processor subsystem, an axial conductor and a low-loss dielectric material, constructed so as to be positioned within said outer conductor and maintained in a fixed position, said distal end in contact with said tissue region being interrogated;
a cable jacket constructed so as to surround said outer conductor to preserve transceiver structural integrity and protect said transceiver from environmental elements;
said transceiver constructed so as to match impedances equal to 50 ohms;
said transceiver forming an integrated open-ended co-axial structure;
said transceiver constructed so as to minimize energy loss that occurs at connections and maintains an aspect ratio that minimizes impedance mismatch.

19. A detection system as recited in claim 18 wherein said low-loss dielectric comprises Teflon and wherein said outer conductors and said axial conductors further comprise a material selected from the group consisting of brass, copper, silver gold or nanotubes.

20. A detection system as recited in claim 6 wherein said transceiver comprises:
a hollow outer conductor having a distal end and a proximal end, said proximal end connected to said control and processor subsystem, a continuously tapered axial conductor and a low-loss dielectric material, constructed so as to be positioned within said outer conductor and maintained in a fixed position, said distal end in contact with said tissue region being interrogated;
said tapered axial conductor constructed so as to provide a diminishing ratio between a constant inner diameter of said hollow outer conductor and said continuously increasing diameter of axial conductor so as to continuously decrease component impedance of a transmission line thereby allowing a better impedance match between the detector system and the tissue region being interrogated;
a cable jacket constructed so as to surround said outer conductor to preserve transceiver structural integrity and protect said transceiver from environmental elements;
said transceiver constructed so as to match impedances greater than 50 ohms;
said transceiver forming an integrated open-ended co-axial structure; and
said transceiver constructed so as to minimize energy loss that occurs at connections and maintain an aspect ratio that minimizes impedance mismatch.

21. A detection system as recited in claim 20 wherein said low-loss dielectric comprises Teflon and wherein said outer conductors and said axial conductors further comprise a material selected from the group consisting of brass, copper, silver, gold or nanotubes.

22. A detection system as recited in claim 6 wherein said transceiver comprises:
a hollow flared outer conductor having a distal end and a proximal end, said proximal end connected to said control and processor subsystem, a continuously tapered axial conductor and a low-loss dielectric material, constructed so as to be positioned within said outer conductor and maintained in a fixed position, said distal end in contact with said tissue region being interrogated;
said continuously tapered axial conductor having a continuously increasing diameter from said distal end to said proximal end;
a cable jacket constructed so as to surround said outer conductor to preserve transceiver structural integrity and protect said transceiver from environmental elements;
said transceiver constructed so as to match impedances greater than 50 ohms;
said transceiver forming an integrated open-ended co-axial structure; and
said transceiver constructed so as to minimize energy loss that occurs at connections and maintain an aspect ratio that minimizes impedance mismatch.

23. A detection system as recited in claim 22 wherein said low-loss dielectric comprises Teflon and wherein said outer conductors and said axial conductors further comprise a material selected from the group consisting of brass, copper, silver, gold or nanotubes.

24. A detection system as recited in claim 6, wherein said transceiver comprises:
a hollow outer conductor having a distal end and a proximal end, said proximal end connected to said control and processor subsystem, a flared axial conductor and a low-loss dielectric material, constructed so as to be positioned within said outer conductor and maintained in a fixed position, said distal end in contact with said tissue region being interrogated;
said flared axial conductor constructed so as to have a continuously decreasing diameter from said distal end to said proximal end in connection with said processor subsystem;
a cable jacket constructed so as to surround said outer conductor to preserve transceiver structural integrity and protect said transceiver from environmental elements;
said transceiver constructed so as to match impedances less than 50 ohms;
said transceiver forming an integrated open-ended co-axial structure; and
said transceiver constructed so as to minimize energy loss that occurs at connections and maintain an aspect ratio that minimizes impedance mismatch.

25. A detection system as recited in claim 24 wherein said low-loss dielectric comprises Teflon and wherein said outer conductors and said axial conductors further comprise a material selected from the group consisting of brass, copper, silver, gold or nanotubes.

26. A detection system as recited in claim 6, wherein said transceiver comprises:
a hollow flared outer conductor having a distal end and a proximal end, said proximal end connected to said control and processor subsystem, a flared axial conductor and a low-loss dielectric material, constructed so as to be positioned within said outer conductor and maintained in a fixed position, said distal end in contact with said tissue region being interrogated;
said continuously flared outer conductor and flared axial conductor constructed so as to have a continuously decreasing diameter from said distal end to said proximal end that is in connection with said processor subsystem; a cable jacket constructed so as to surround said outer conductor to preserve transceiver structural integrity and protect said transceiver from environmental elements;
said transceiver constructed so as to match impedances less than 50 ohms;
said transceiver forming an integrated open-ended co-axial structure; and
said transceiver constructed so as to minimize energy loss that occurs at connections and maintain an aspect ratio that minimizes impedance mismatch.

27. A detection system as recited in claim 26 wherein said low-loss dielectric comprises Teflon and wherein said outer conductors and said axial conductors further comprise a material selected from the group consisting of brass, copper, silver, gold or nanotubes.

28. A detection system as recited in claim 1, wherein said system is a hand-held device.

29. A detection system as recited in claim 6, wherein said wand-detector comprises control switches and tactile feedback including but not limited to temperature and vibration sensors that allow for user input and feedback.
